(19) United States
(12) Patent Application Publication
(10) Pub. No.: US 2020/0237980 A1
(43) Pub. Date: Jul. 30, 2020

(54) ACTUATOR SYSTEM WITH VIRTUAL AND PHYSICAL PORTIONS

(71) Applicant: The Regents of the University of California, Oakland, CA (US)

(72) Inventor: Daisuke Sato, Davis, CA (US)

(21) Appl. No.: 16/753,107

(22) PCT Filed: Oct. 4, 2018
(86) PCT No.: PCT/US2018/054430

§ 371 Date: Apr. 2, 2020

Related U.S. Application Data
(60) Provisional application No. 62/568,160, filed on Oct. 4, 2017.

(51) Int. Cl. A61M 1/10

(52) U.S. Cl. CPC A61M 1/1055; A61M 2205/0244; A61M 1/1068; A61M 1/1058

(57) ABSTRACT

An actuation system includes a virtual or computer-modeled portion that is coupled to a physical portion. The virtual portion is a computer model that models or otherwise simulates a function or action, such as a physiological function or action, including for example an action potential, a calcium transient, and/or a chemical reaction. The computer model may model or simulate a chemical action, a mechanical action (such as movement of a wing) or any other action. The virtual portion drives or controls one or more physical actuators, which can be sized on a microscopic scale, such as on a nanometer scale. The actuation system can be used as or part of an artificial anatomical structure or organ, such as an artificial heart.

This invention was made with Government support under Grant No. R00-HL111334, awarded by the National Institutes of Health. The Government has certain rights in this invention.

### BACKGROUND

There is an ongoing effort to build mechanical systems that elegantly mimic or simulate the mechanisms that occur in nature. For example, artificial hearts are configured to mimic the movement and action of the natural heart. Another example is in the field of robotics where there is a growing effort to accurately simulate incremental movements and motion that occur in the natural skeletal system and muscular systems.

Such artificial systems can be severely limited by the mechanical actuation system that control the artificial, physical structure such as an artificial heart or muscle. Current actuation systems are typically limited by both the size of the mechanical portion of the actuator as well as the control signal that governs the movement of actuator. A mechanical actuator can be relatively large in size, which limits the amount of incremental and fine-level movement that can be achieved by the actuator. Although microscopic microelectromechanical systems can somewhat overcome the size limitations of traditional mechanical actuators, such systems are still limited by the control signal that governs movement of the actuator.

In view of the foregoing, there is a need for improved systems and methods for accurately simulating the mechanical and control systems that occur in nature.

### SUMMARY

Disclosed is an actuator system (or actuation system) that includes a computational model that drives or controls a physical actuator. In a non-limiting embodiment, the actuator can serve as an artificial organ or muscle, as described below. For example, the actuator can serve as an artificial muscle with computational model performing a virtual action potential and a virtual calcium transient, wherein the computational model controls a physical, mechanical contraction unit that mimics movement of a muscle. In other embodiments, the actuator system can achieve deformations and/or movements of a dynamic structure, such as an automobile, aircraft, spacecraft, watercraft. The actuator system can also achieve deformations and/or movements of a static structure, such as a chair, desk, building, etc. The actuation system can achieve such deformations or movements via a physical, chemical, mechanical, and/or electrical dynamics or combinations thereof. The model provides an output that drives a change in the physical structure wherein the change is achieved or accomplished via a physical, chemical, mechanical, and/or electrical dynamic or combinations thereof.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 shows a schematic representation of an actuation system.

FIG. 2 shows another schematic representation of an actuation system that models an actuation potential and calcium transient.

FIG. 3 shows a schematic representation of a collection of actuation systems that form a collection of virtual cells.

FIG. 4 shows a collection of virtual cells each with an onboard computational unit.

FIG. 5 shows a collection of virtual cells each with an outboard computational unit.

FIGS. 6 and 7 show an example of a fiber formed of cells that include one or more actuation systems formed of electromagnets.

FIGS. 8 and 9 show an example of a collection of fibers embedded in an elastic body.

FIGS. 10 and 11 show an example of a fiber formed of cells that include one or more actuation systems formed of ultrasonic motors.

FIG. 12 shows a section of shape memory alloy having a collection of actuation system cells embedded therein.

FIG. 13 shows an example of an electrostatic actuator.

FIG. 14 shows an example of an electrostatic linear motor.

### DETAILED DESCRIPTION

Disclosed herein is an actuation system that includes a virtual or computer-modeled portion that is coupled to a physical portion. In an embodiment, the virtual portion is a computer model that models or otherwise simulates a function or action, such as a physiological function or action, including for example an action potential, a calcium transient, and/or a chemical reaction, as described more fully below. It should be appreciated that the computer model is not limited to simulating a physiological function but can model or simulate other actions or functions. For example, the computer model may model or simulate a chemical action, a mechanical action (such as movement of a wing) or any other action. The virtual portion drives or controls one or more physical actuators, which can be sized on a microscopic scale, such as on a nanometer scale. The actuation system can be used as or part of an artificial anatomical structure or organ, such as an artificial heart, muscle, bone, etc.

In a non-limiting example, the actuation system is configured to model cardiac function and can be part of an artificial heart or be coupled to a natural heart. The actuation system can comprise an artificial cardiac muscle actuator with a combination of the virtual action potential, virtual calcium transient, and physical mechanical contraction unit using high performance computing and microelectromechanical systems. The artificial cardiac muscle actuator can include or be coupled to natural cardiac cells and/or tissue. The actuation system can receive electrical signals from natural cardiac cells and tissue and physically contract based on a real time computer model and emit electrical signals to effectuate contraction of a natural heart.

To the extent that the actuation artificial cardiac muscle actuator includes or is coupled to cardiac cells and/or tissue, the corresponding cardiac cells/tissues can be Induced Pluripotent Stem Cells (iPS) or iPS derived cardiac cells/tissue. In addition, the actuation system, when implanted in a heart, can stimulate surrounding heart tissue to reshape the natural action potential and/or act as an additional pace making site to prevent or suppress arrhythmias.

To make artificial cardiac tissue a single cell unit can be used to develop cardiac tissue using multi cellular units. The virtual portion (as described below) can use, for example, a physiologically detailed action potential model that can reproduce physiological action potentials and Calcium transients. The physical actuator or unit can be any of a variety of physical units as described herein. To make artificial cardiac tissue, the cellular units can be developed into cardiac muscle tissue actuators. Tissue actuators such as 1 dimensional, two dimensional, and three dimensional actuators can be used. A one dimensional actuator can be formed of a single string in series of cellular units embedded in elastic bodies. Non-limiting examples of elastic bodies include silicone rubber and sodium alginate. A two dimensional actuator can be formed of a planar or sheet assembly of one dimensional actuators, while a three dimensional actuator can be formed of a stack of sheets of two dimensional actuators.

Each cellular unit is independent and receives signals of contraction generated by a computer for example. Electrical power is delivered to each cellular unit and a feedback signal comprised of a physical displacement (such as contraction) and/or force of each cell is used. The virtual action potential of healthy and diseased conditions can be reflected by the physical displacement or contraction.

In other non-limiting example embodiments, the actuation system can be used to achieve deformations or shape changes in structures such as automobiles, aircraft, spacecraft, watercraft, buildings, and static objects such desks and chairs.

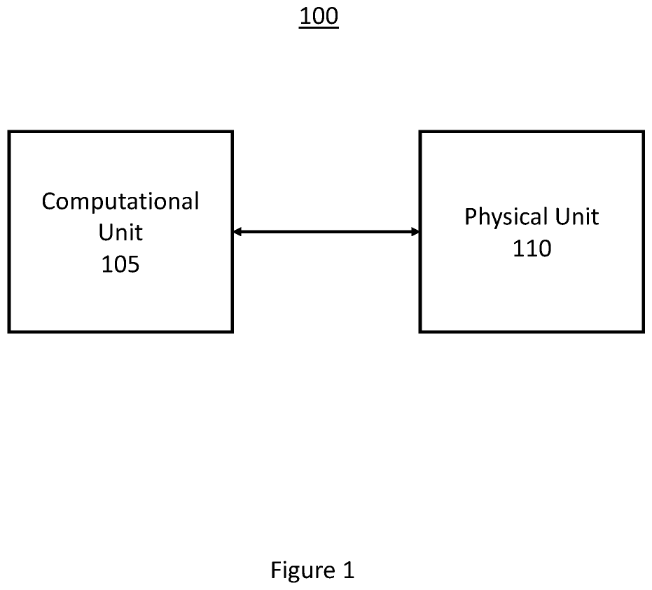

FIG. 1 shows a schematic representation of an actuation system 100 that has a virtual portion comprising a computational unit 105 that is communicatively coupled to a physical unit 110. The computational unit 105 is computer-implemented model that simulates a physiological action, as described below. The physical unit 110 is a physical actuator that has a microscopic size, such as on a nanoscale. In an embodiment, the physical unit is or includes a microelectromechanical system (MEMS.) The computational unit 105 can be communicatively and/or electrically coupled to the physical unit 110 via a wired or wireless connection that provides two-way communication between the two units. The computational unit 105 provides an output that controls the physical unit 110. The physical unit 110 can also provide a feedback signal to the computational unit, wherein the feedback signal can include mechanical data such as a level of deformation or movement of the physical unit.

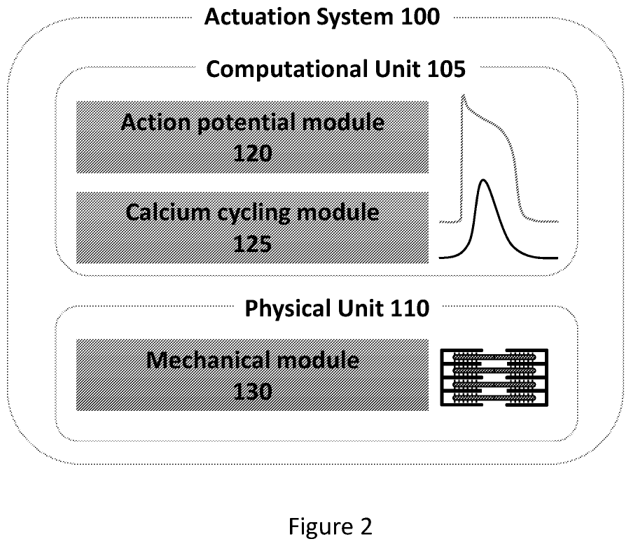

FIG. 2 shows an example embodiment of the actuation system 100 wherein the computational unit 105 is or includes a computational model of one or more physiological functions. In the illustrated embodiment, the computational models include an action potential module 120 and a calcium cycling module 130. The computational unit drives or otherwise controls a mechanical module 130, which may be a microelectromechanical system that forms or is part of a biological cell structure or collection of cell structures, as described further below.

With reference still to FIG. 2, the action potential module is a computational model that simulates the processes of ion channels, cell membrane, organelle of the action potential physiological function. The calcium cycling module is a computational module that simulates the intercellular calcium signaling process. In this manner, the computational unit reproduces, mimics, simulates, or otherwise virtually models physiological action potential and calcium transient processes to output a command to the mechanical module 130 to cause the mechanical module to change shape in a manner that mimics the movement (i.e., contraction, expansion, rotation, or combinations thereof) of a biological or anatomical structure, such as a muscle. For example, the computational unit 105 can simulate in real time the action potential and calcium transient processes to control the mechanical module in a manner that mimics or simulates the contraction of an artificial muscle such as the heart muscle and/or skeletal muscles.

The manner in which the computational unit models the action potential and/or the calcium transients can vary. In an example, the action potential model is performed pursuant to the computer model. In a non-limiting embodiment, mathematical modeling of the cardiac action potential includes modeling of cardiac arrhythmias and/or other functions of an animal heart such as a human heart. The model can reproduce the dynamics of the cardiac action potential and intracellular calcium cycling at rapid heart rates such as relevant to ventricular tachycardia and fibrillation. In a non-limiting embodiment, the model is at least partially based on a rabbit ventricular action potential model wherein L-type calcium current and intracellular calcium cycling formulations are modified based on experimental patch-clamp data obtained in isolated rabbit ventricular myocytes using perforated patch configurations at 35 degrees Celsius for example. A minimal seven-state Markovian model can be incorporated that reproduces Calcium and voltage dependent kinetics in combination with existing dynamic intracellular Calcium cycling models to replicate action potential duration and intracellular transient alternans at rapid heart rates and also reproduces experimental action potential duration restitution curves obtained by either dynamics or S1S2 pacing. The following publication, which is incorporated by reference, provides details regarding a physiologically detailed computer model of the rabbit ventricular myocyte in two-dimensional tissue to determine how spiral waves respond to β-adrenergic activation following administration of isoproternol: “How does β-adrenergic signalling affect the transitions from ventricular tachycardia to ventricular fibrillation?” published in European Society of Cardiology, Europace (2014) 16, 452-457 doi:10.1093/europace/eut412 by Yuanfang Xie, Eleonora Grandi, Donald M. Bers, and Daisuke Sato, which is incorporated herein by reference in its entirety.

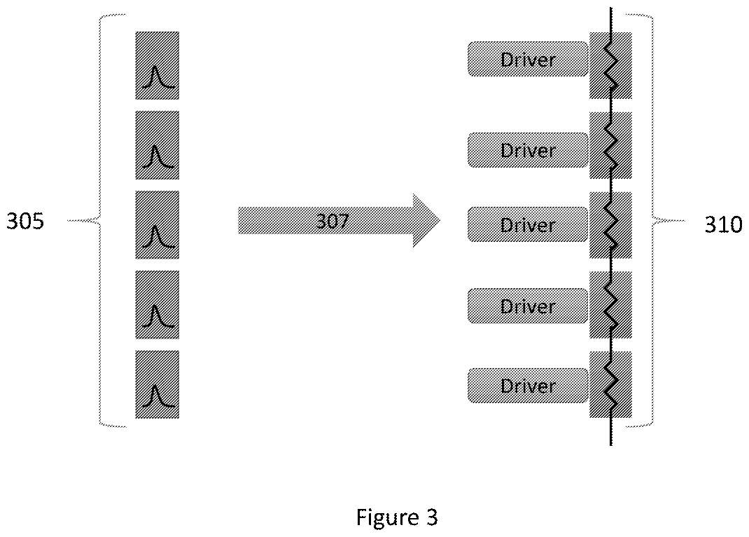

As mentioned, the actuation system 100 can have a size on a microscopic scale such as on a nanometer˜millimeter scale, which is a non-limiting example. In this regard, each actuation system can be smaller in size than a biological cell structure such as smaller than the scale of organelle of a cell. A series or collection of actuation systems 100 can be assembled to form a collection of virtual cells 305, such as shown in FIG. 3. Each virtual cell can include one or more actuation systems 100 of the type represented in FIG. 2. The collection of virtual cells provides a digital output 307, which can be converted to an analog signal to drive a corresponding set of physical actuators 310. The output 307 can be communicated via wire or wirelessly to the physical actuators 310. The output drives a mechanical change, such as a contraction, expansion, rotation, or any other change in shape, of the physical actuators 310. The physical actuators can be coupled to or can form a surface of a structure such as an artificial heart or artificial muscle to achieve changes in shape of the structure.

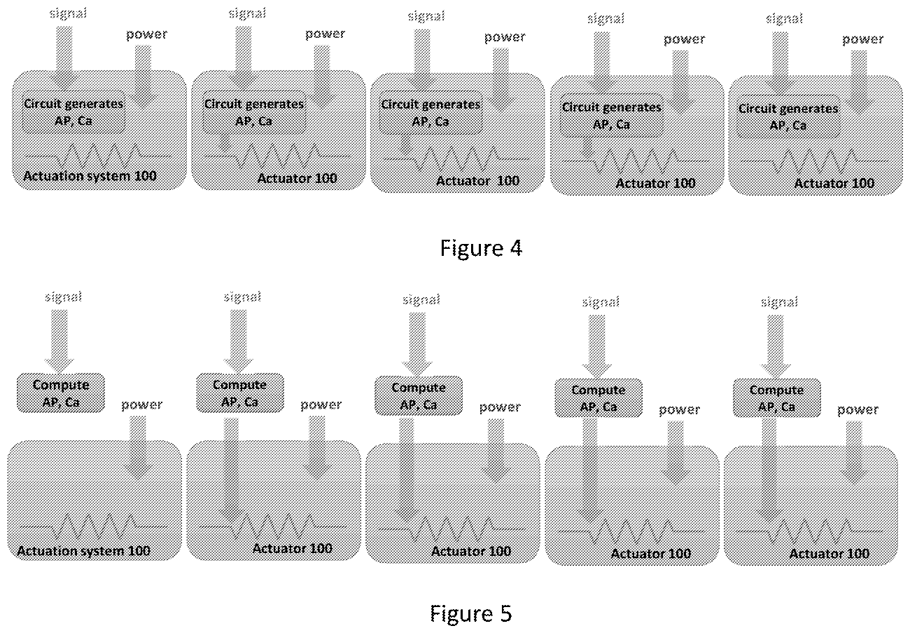

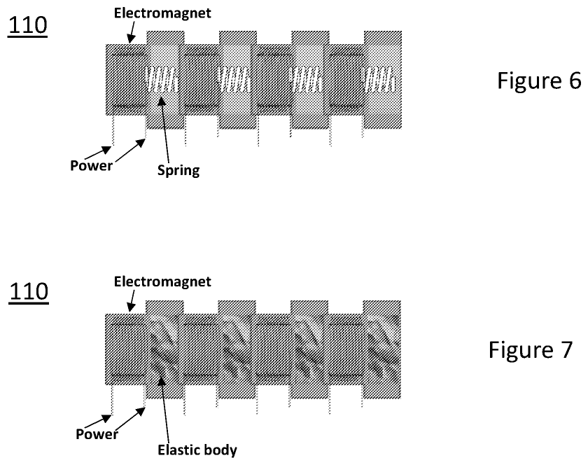

The actuation system 100 or a collection of actuation systems 100 can include a power supply. FIG. 4 shows a collection or series of actuation systems 100 (or actuators), wherein each actuation system 100 represents a cellular unit. As shown in FIG. 4, each actuation system 100 has an external power supply. In a non-limiting example, the power supply supplies electrical power from the outside of the cellular module. It should be appreciated that the power supply can provide power other than electrical power. An internal electrical circuit for each actuation system 100 can be used to generate the computational model of the action potential and calcium transient. In another embodiment shown in FIG. 5, the actuation systems each have an external computing unit for generating the computational model of the action potential and calcium transient.

In use, the cells (each of which includes one or more actuation systems 100) can be embedded or otherwise coupled to a structural body that can change in shape in response to the virtual action potential and virtual calcium transient that drives the physical actuator. The structural body can be a one-dimensional fiber or a planar body in which the cells are arranged in a two-dimensional plane or sheet. The structural body can also be a three-dimensional arrangement formed of a stacked collection of two-dimensional sheets. In any event, each cell is independent and can receive signals for contraction and electric power. The cells can also provide a feedback signal, such as displacement or movement of each cell and a controller can be used to adjust further movement based on the feedback signal. The cells collectively form an artificial tissue that can achieve changes in shape, size, stiffness, or other mechanical property based on the simulated action potential and calcium transient.

As mentioned, the actuation system 100 can also be used to achieve or effectuate mechanical changes in other structures, such as the shape of a vehicle (including an automobile, aircraft, watercraft, etc.) The actuation system 100 can also achieve structural changes in passive or static structures, such as the shape of a chair, desk, mobile phone or any ergonomic shape of any structure.

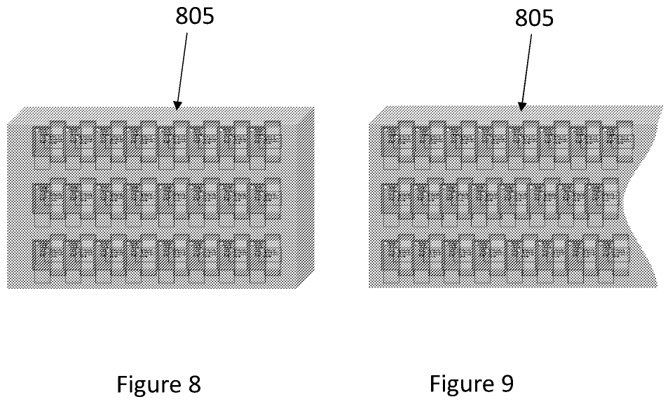

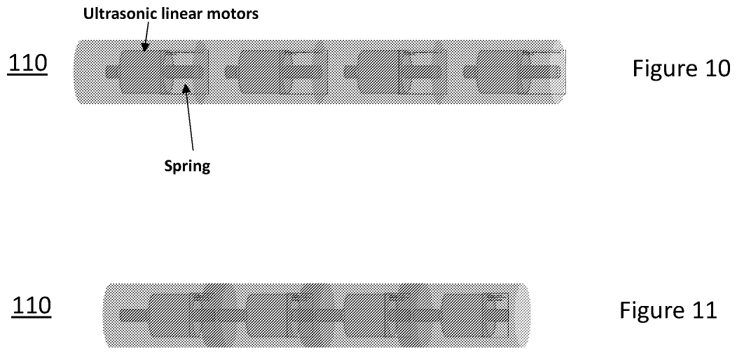

There are now described various examples of mechanical structures that can serve as the physical unit 110 of the actuation system. In a first example shown in FIGS. 6-7, the physical unit 110 is at least partially formed of an electromagnet. A one-dimensional fiber can be assembled by connecting multiple cells or cellular unit, each of which includes an actuation system. In the example of FIG. 6, the fiber includes four cells although the quantity can vary. Each cell can be controlled independently such that each cell in the fiber can contract simultaneously or independently. The cells can be connected by a biased mechanical structure such as a spring (as shown in FIG. 6) or an elastic body (as shown in FIG. 7) that is biased to return the structure to a default position or shape. In addition, a series or collection of such fibers can be collectively embedded in soft elastic body 805 (as shown in FIG. 8) that can deform in shape (as shown in FIG. 9) based upon the action of the actuation systems in the fiber.

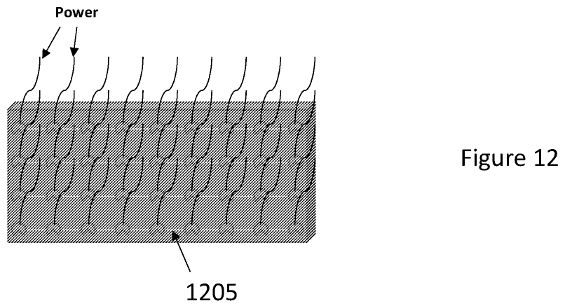

In another example of the physical unit 110, the physical unit 110 is at least partially formed of an ultrasonic linear motor. As shown in FIGS. 10 and 11, each cell includes a physical unit 110 comprising an ultrasonic linear motor with a mechanical structure such as a spring positioned between each cell. As shown in FIG. 11, the physical units 110 can change shape such as by contracting based upon the input of the computational unit.

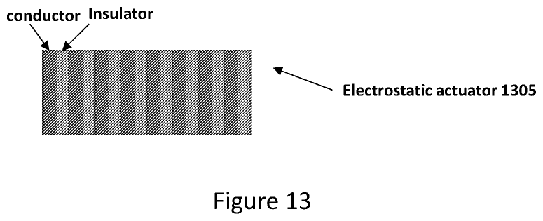

In another example of the physical unit 110, the physical unit 110 is at least partially formed of a shape memory alloy (SMA) (such as an SMA manufactured by TOKI Corp.) which can contract in only one direction. FIG. 12 shows a section of SMA 1205 having a collection of actuation system cells embedded therein. The shape change can be achieved by controlling the actuation system of each cell using a power signal that represents the computational unit of the actuation system 100.

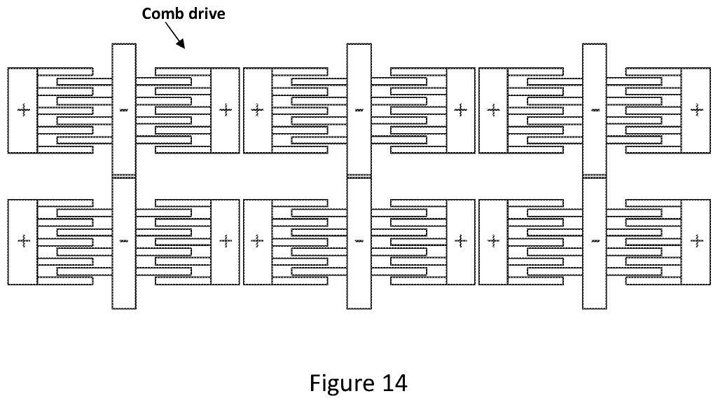

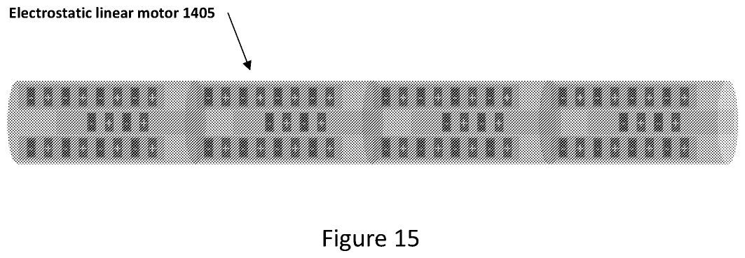

In another example of the physical unit 110, the physical unit 110 is at least partially formed of an electrostatic actuator 1305 used for contraction. As shown in FIG. 13, a series of elastic insulators has a corresponding series of sandwiched conductors that can collectively expand or contact based upon an input signal from the computational unit of the actuator system. In another embodiment, shown in FIG. 14, a series of comb drives embedded in soft elastic body can collectively expand or contract based upon an input signal from the computational unit of the actuator system. In another embodiment, shown in FIG. 15, the physical unit 110 is at least partially formed of an electrostatic linear motor 1405 that can expand and contract in shape based upon an input signal from the computational unit of the actuator system

In another non-limiting example, the physical unit 110 is at least partially formed of a piezoelectric element.

As mentioned, the actuation system can be used to achieve movement, shape change, or deformation of any of a variety of structures. For example, the system can be used for artificial organs such as an artificial heart or prostheses. The system can be used for artificial muscle in robots including humanoid robots. This actuator system can also provide an alternative method of iPS cell-engineered cardiac tissue for cardiac regeneration. In addition, embedded artificial cardiac tissue can pace the tissue and actively suppress arrhythmias by off-site pacing. The actuator system can be used to effectuate a more natural, more realistic, and more comfortable prosthesis.

The actuation system can also be used in vehicles such as automobiles, airplanes, rockets, ships and submarines. The actuation system can be used to improve maneuverability of such vehicles by mimicking shape changes that occur in natural biological processes or organisms. The actuation system can also be used to achieve “smart” shape changes in structures such as buildings by controlling material properties such as the stiffness of the materials that form the structure.

One or more aspects or features of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system. The implementations can also include at least one input device (e.g., mouse, touch screen, etc.) and at least one output device.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, the subject matter described herein can be implemented on a computer having a computer processor and a display device, such as for example a cathode ray tube (CRT) or a liquid crystal display (LCD) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In an example, a prototype actuation system 100 was built that included a shape memory alloy wrapped in a silicone wrapper. A single board computer comprised of a Raspberry Pi computer was used to program the prototype actuation system 100.

As will be apparent to those of skill in the art upon reading this disclosure, each of the individual embodiments described and illustrated herein has discrete components and features which may be readily separated from or combined with the features of any of the other several embodiments without departing from the scope of the subject matter described herein. Any recited method can be carried out in the order of events recited or in any other order which is logically possible.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

1. An actuation system, comprising:
a computational model that simulates a function;
a physical structure communicatively coupled to the computational model, wherein the computational model provides an output that drives a change in the physical structure.

2. The actuation system of claim 1, wherein the computational model simulates a physiological function.

3. The actuation system of claim 1, wherein the computational model simulates at least one of an action potential and a calcium transient.

4. The actuation system of claim 1, wherein the physical structure is an electromagnet.

5. The actuation system of claim 1, wherein the physical structure is an ultrasonic linear motor.

6. The actuation system of claim 1, wherein the physical structure is a shape memory alloy.

7. The actuation system of claim 1, wherein the physical structure is an electrostatic actuator.

8. The actuation system of claim 1, wherein the physical structure is an electrostatic linear motor.

9. The actuation system of claim 1, wherein the physical structure is a piezoelectric element.

10. The actuation system of claim 1, wherein the computational model communicates with the physical structure via a wired or a wireless connection.

11. The actuation system of claim 1, wherein the physical change is at least one of a contraction, an expansion, a displacement, and a change in stiffness.

12. The actuation system of claim 1, wherein the physical structure is a microelectromechanical system.

13. The actuation system of claim 1, wherein the change in the physical structure is at least one of a physical, chemical, mechanical, and/or electrical change.

14. The system of claim 1, wherein the computational model simulates a function of a human heart.

15. The system of claim 14, wherein the physical structure is an artificial or natural heart, and wherein the change in the physical structure is a contraction.

16. The system of claim 13, wherein the computational model simulates at least one of an action potential and a calcium transient.

17. The system of claim 1, further comprising a natural or artificial heart.

18. The system of claim 17, further comprising at least one of an artificial cardiac cell and an artificial cardiac tissue.
